(19) United States
(12) Patent Application Publication
(10) Pub. No.: US 2017/0283455 A1
(43) Pub. Date: Oct. 5, 2017

(54) STABLE TOPICAL PHAMACEUTICAL COMPOSITIONS OF HALOBETASOL PROPIONATE

(71) Applicant: SUN PHARMACEUTICAL INDUSTRIES LIMITED, Mumbai (IN)

(72) Inventors: Anil Rana, Chandigarh (IN); Lav KESHRI, Lucknow (IN); Simrata BEDI, Gurgaon (IN); Ravi KOCHHAR, Gurgaon (IN); Sairab KHAN, Gurgaon (IN); Kaushal NAYYAR, Gurgaon (IN); Mohan PRASAD, Gurgaon (IN)

(21) Appl. No.: 15/618,479

(22) Filed: Jun. 9, 2017

Related U.S. Application Data
(63) Continuation-in-part of application No. 15/024,977, filed on Mar. 25, 2016.
Application No. 15/024,977 is a continuation-in-part of application No. 15/024,984, filed on Mar. 25, 2016.

(30) Foreign Application Priority Data
Sep. 25, 2013 (IN) 2838/DEL/2013
Sep. 25, 2013 (IN) 2839/DEL/2013

(51) Int. Cl. C07J 13/00; A61K 9/00; A61K 47/10; A61K 31/573

(52) U.S. Cl. CPC C07J 13/007; A61K 31/573; A61K 9/0014; A61K 47/10

(57) ABSTRACT

The present invention provides a stable topical pharmaceutical composition of halobetasol propionate. The present invention discloses an impurity of compound of Formula II and its process of preparation. It also relates to a method of evaluating stability of a topical pharmaceutical composition of halobetasol propionate.

### RELATED APPLICATIONS

This application is a continuation-in-part of U.S. Ser. No. 15/024,977, filed Mar. 25, 2016, which is a 371 filing of PCT/IB2014/064745, filed Sep. 22, 2014 claiming priority to IN2839/DEL/2013, filed Sep. 25, 2013, and a continuation-in-part-of U.S. Ser. No. 15/024,984, a 371 filing of PCT/IB2014/064802, filed Sep. 24, 2014, claiming priority to IN2838/DEL/2013, filed Sep. 25, 2013, each of which is incorporated by reference in their entirety.

### FIELD OF THE INVENTION

The present invention provides a stable topical pharmaceutical composition of halobetasol propionate. The present invention discloses an impurity of compound of Formula II and its process of preparation. It also relates to a method of evaluating stability of a topical pharmaceutical composition of halobetasol propionate.

### BACKGROUND OF THE INVENTION

Halobetasol propionate chemically is 21-chloro-6α,9-difluoro-11β,17-dihydroxy-16β-methylpregna-1,4-diene-3,20-dione, 17-propionate, represented by Formula I.

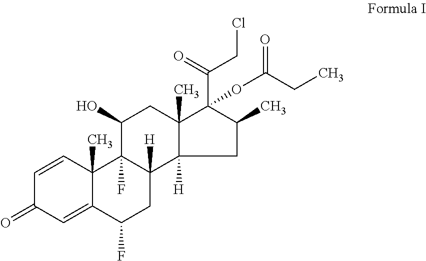

Halobetasol propionate is a super-potent corticosteroid widely used for the treatment of dermal ailments. Topical dosage forms of halobetasol propionate, such as cream, lotion, and ointments, are commercially available under the trade name Ultravate® and have been indicated for the relief of inflammatory and pruritic manifestations of corticosteroid-responsive dermatoses.

There is a need for a topical pharmaceutical composition of halobetasol propionate with improved stability with respect to the formation of impurities during storage. The efficacy and safety of a pharmaceutical product gets affected by the presence of any impurity even in trace amount whether present initially or generated during storage of the product. Identification of impurities during manufacture of dosage forms becomes important for determining the stability of the product and hence the shelf-life of pharmaceutical product. The inventors of the present invention have surprisingly observed that an impurity represented by Formula II is generated in the topical pharmaceutical composition of halobetasol propionate when a particular solvent is selected in the preparation of the topical pharmaceutical composition. By controlling it to an acceptable level, the stability of the topical pharmaceutical composition of halobetasol propionate can be ensured.

### SUMMARY OF THE INVENTION

The present invention provides a stable topical pharmaceutical composition of halobetasol propionate. The present disclosure is directed to a compound of Formula II which can act as an impurity that reduces stability of certain topical pharmaceutical compositions of halobetasol propionate. It also relates to a method of evaluating stability of a topical pharmaceutical composition of halobetasol propionate by controlling its level in the composition to a particular level. This invention is directed in part to a marker that can assist in the preparation of a stable pharmaceutical composition of halobetasol propionate.

### DETAILED DESCRIPTION OF THE INVENTION

A first aspect of the present invention provides a compound of Formula II

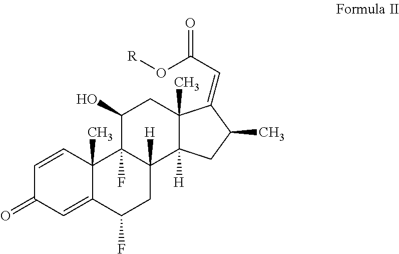

wherein R is C₁-C₄ alkyl.

According to one embodiment of the above aspect, the compound of Formula II, wherein R is ethyl.

According to another embodiment of the above aspect, the compound of Formula II is in isolated form.

A second aspect of the present invention provides a process for the preparation of a compound of Formula II, comprising reacting halobetasol propionate of Formula I with a non-aqueous solvent, and wherein the Formula II is represented as

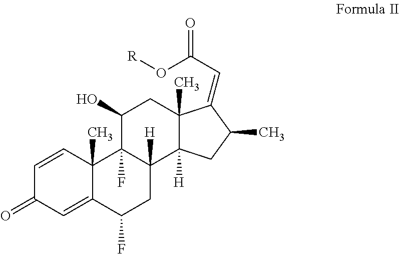

wherein R is C₁-C₄ alkyl.

According to one embodiment of the above aspect, the compound of Formula II, wherein R is ethyl.

According to another embodiment of the above aspect, the non-aqueous solvent is selected from the group consisting of methyl alcohol, ethyl alcohol, isopropyl alcohol, n-propyl alcohol, isobutyl alcohol, n-butyl alcohol, or mixtures thereof. In a preferred embodiment of the above aspect, the non-aqueous solvent is ethyl alcohol. In a more preferred embodiment of the above aspect, the non-aqueous solvent is dehydrated ethyl alcohol

The compound of Formula II may optionally be isolated by filtration, decantation, extraction, distillation, evaporation, chromatography, precipitation, concentration, crystallization, centrifugation, and recrystallization. The compound of Formula II may be dried using conventional techniques, for example, drying, drying under vacuum, spray drying, air drying, or agitated thin film drying.

A third aspect of the present invention provides use of a compound of Formula II as a reference standard in the analysis of halobetasol propionate, and wherein the Formula II is represented as

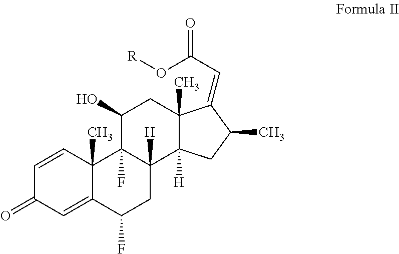

wherein R is C₁-C₄ alkyl.

According to one embodiment of the above aspect, the compound of Formula II, wherein R is ethyl.

A fourth aspect of the present invention provides a high purity halobetasol propionate, wherein the content of a compound of Formula II is not more than 2.0% w/w based on the total weight, and wherein the Formula II is represented as

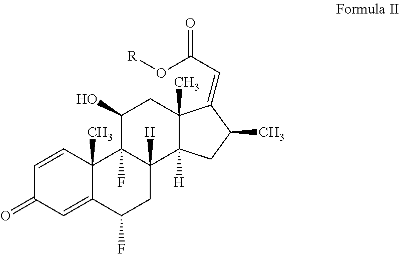

wherein R is C₁-C₄ alkyl.

According to one embodiment of the above aspect, the compound of Formula II, wherein R is ethyl.

According to another embodiment of the above aspect, the content of the compound of Formula II is not more than 1.0% w/w based on the total weight.

According to another embodiment of the above aspect, the content of the compound of Formula II is not more than 0.5% w/w based on the total weight.

A fifth aspect of the present invention provides a stable topical pharmaceutical composition of halobetasol comprising halobetasol propionate and a compound of Formula II, wherein the content of a compound of Formula II is not more than 2.0% w/w based on the total weight of the composition, and wherein the Formula II is represented as

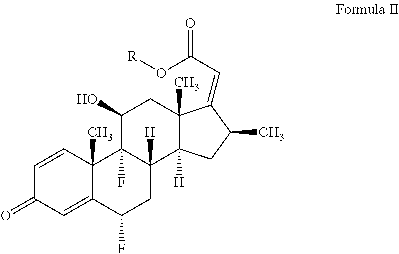

wherein R is C₁-C₄ alkyl.

According to one embodiment of the above aspect, the compound of Formula II, wherein R is ethyl.

According to another embodiment of the above aspect, the content of the compound of Formula II is not more than 1.0% w/w based on the total weight of the composition.

According to another embodiment of the above aspect, the content of the compound of Formula II is not more than 0.5% w/w based on the total weight of the composition.

A sixth aspect of the present invention provides a stable topical pharmaceutical composition of halobetasol comprising halobetasol propionate, a non-aqueous solvent, and a compound of Formula II, wherein the content of a compound of Formula II is not more than 2.0% w/w based on the total weight of the composition, and wherein the Formula II is represented as—

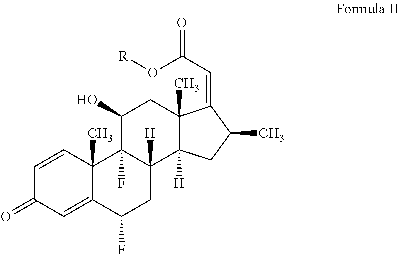

wherein R is C₁-C₄ alkyl.

According to one embodiment of the above aspect, the compound of Formula II, wherein R is ethyl.

According to another embodiment of the above aspect, the content of the compound of Formula II is not more than 1.0% w/w based on the total weight of the composition.

According to another embodiment of the above aspect, the content of the compound of Formula II is not more than 0.5% w/w based on the total weight of the composition.

According to another embodiment of the above aspect, the non-aqueous solvent is selected from the group consisting of methyl alcohol, ethyl alcohol, isopropyl alcohol, n-propyl alcohol, isobutyl alcohol, n-butyl alcohol, or mixtures thereof. In a preferred embodiment of the above aspect, the non-aqueous solvent is ethyl alcohol. In a more preferred embodiment of the above aspect, the non-aqueous solvent is dehydrated ethyl alcohol

According to another embodiment of the above aspect, the non-aqueous solvent is present in an amount of 10% w/w to about 60% w/w based on the total weight of the composition. Particularly, the non-aqueous solvent is present from about 20% w/w to about 50%, more particularly, the non-aqueous solvent is present from about 35% w/w to about 45% w/w based on the total weight of the composition. Even, more particularly, non-aqueous solvent is present from about 38% w/w to about 43% w/w based on the total weight of the composition.

According to another embodiment of the above aspect, the halobetasol propionate is present from about 0.01% w/w to about 0.5% w/w based on the total weight of the composition. Particularly, the halobetasol propionate is present in 0.05% w/w based on the total weight of the composition.

According to another embodiment of the above aspect, the composition is stored at 25° C. and 60% Relative humidity (RH) for at least 3 months. More particularly, the composition is stored at 25° C. and 60% RH for at least 6 months.

According to another embodiment of the above aspect, the composition is stored at 40° C. and 75% RH for at least 3 months. More particularly, the composition is stored at 40° C. and 75% RH for at least 6 months.

A seventh aspect of the present invention provides a method for evaluating stability of a topical pharmaceutical composition of halobetasol propionate comprising:
(a) a step of dissolving halobetasol propionate in a non-aqueous solvent to form a solution;
(b) optionally mixing one or more pharmaceutically acceptable excipients; and
(c) a step of measuring the amount of a compound of Formula II;
   wherein the content of the compound of Formula II is not more than 2.0% w/w based on the total weight of the composition, and wherein the Formula II is represented as

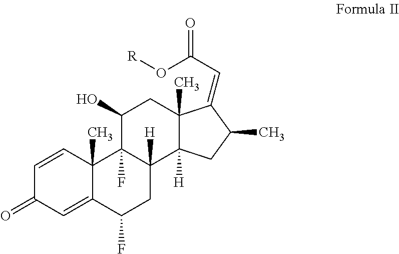

wherein R is C₁-C₄ alkyl.

According to one embodiment of the above aspect, the compound of Formula II, wherein R is ethyl.

According to another embodiment of the above aspect, the content of the compound of Formula II is not more than 1.0% w/w based on the total weight of the composition.

According to another embodiment of the above aspect, the content of the compound of Formula II is not more than 0.5% w/w based on the total weight of the composition.

According to another embodiment of the above aspect, the non-aqueous solvent is selected from the group consisting of methyl alcohol, ethyl alcohol, isopropyl alcohol, n-propyl alcohol, isobutyl alcohol, n-butyl alcohol, or mixtures thereof. In a preferred embodiment of the above aspect, the non-aqueous solvent is ethyl alcohol. In a more preferred embodiment of the above aspect, the non-aqueous solvent is dehydrated ethyl alcohol

According to another embodiment of the above aspect, the non-aqueous solvent is present in an amount of 10% w/w to about 60% w/w based on the total weight of the composition. Particularly, the non-aqueous solvent is present from about 20% w/w to about 50%, more particularly, the non-aqueous solvent is present from about 35% w/w to about 45% w/w based on the total weight of the composition. Even, more particularly, non-aqueous solvent is present from about 38% w/w to about 43% w/w based on the total weight of the composition.

According to another embodiment of the above aspect, the halobetasol propionate is present from about 0.01% w/w to about 0.5% w/w based on the total weight of the composition. Particularly, the halobetasol propionate is present in 0.05% w/w based on the total weight of the composition.

According to another embodiment of the above aspect, the composition is stored at 25° C. and 60% RH for at least 3 months. More particularly, the composition is stored at 25° C. and 60% RH for at least 6 months.

According to another embodiment of the above aspect, the composition is stored at 40° C. and 75% RH for at least 3 months. More particularly, the composition is stored at 40° C. and 75% RH for at least 6 months.

An eight aspect of the present invention provides a method for analyzing a halobetasol composition sample for the presence or amount of an impurity compound represented by Formula II comprising:
(a) providing a halobetasol composition sample comprising halobetasol propionate;
(b) using a spectral or toxicology analysis to determine the presence of an impurity compound represented by Formula II

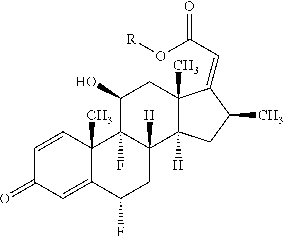

wherein R is ethyl; and
(c) determining the presence of the structural signature associated with the compound of Formula II thereby to analyze the halobetasol composition sample.

According to one embodiment of the above aspect, the spectral analysis comprises analyzing the sample using a liquid chromatography.

According to another embodiment of the above aspect, the liquid chromatography is ultra-performance liquid chromatography.

According to another embodiment of the above aspects, the composition is selected from the group comprising solution, emulsion, gel, lotion, cream, ointment, foam, pastes, or the like. In a preferred embodiment, the composition is a solution. In a more preferred embodiment, the composition is a solution delivered in the form of a spray.

According to another embodiment of above aspect, the composition is administered to a subject in need thereof for treating inflammatory and pruritic manifestations of corticosteroid responsive dermatoses. In a preferred embodiment, the corticosteroid responsive dermatoses includes psoriasis and atopic dermatitis.

According to another embodiment of the above aspect, the composition is substantially anhydrous.

According to another embodiment of the above aspect, the composition is non-foaming.

According to another embodiment of the above aspect, the composition is preservative-free.

According to another embodiment of the above aspects, the composition is stabilizer-free.

The term “topical,” as used herein, refers to a composition meant for application to the skin, scalp, nail, or mucosal tissue.

The term “reference standard,” as used herein, refers to a compound of Formula H that may be used both for quantitative and qualitative analysis of halobetasol propionate. In particular the compound of Formula II is having R as ethyl.

The term “stable,” as used herein, means a compound of Formula II is formed in the topical pharmaceutical composition of halobetasol propionate in an amount of not more than 2.0% w/w based on the total weight of the composition either initially or on storage at 40° C. and 75% RH or at 25° C. and 60% RH for a period of at least one month, particularly three months, more particularly six months to the extent necessary for sale and use of composition. Particularly, the amount of the compound of Formula II is not more than 1.0% w/w based on the total weight of the composition. More particularly, the amount of the compound of Formula II is not more than 0.5% w/w based on the total weight of the composition.

The term “substantially anhydrous,” as used herein means that the content of free water in the composition is not more than 40% w/w, particularly 30% w/w, more particularly not more than 15% w/w, based on the total weight of the composition.

The term “about” as used herein, refers to any value which lies within the range defined by a variation of up to ±10% of the value.

In pharmaceutical industry, assurance must be provided through product development studies that excipients do not adversely affect either the stability of the final product or the availability of the active ingredient at the site of action. The drug in a solubilized form remains perquisite for a formulator as the solubilized drug shows more efficacy for the treatment of skin ailments such as psoriasis and atopic dermatitis. Halobetasol propionate being insoluble in water, alcohol was selected as a solvent of choice to solubilize halobetasol propionate. The use of alcohol, however, surprisingly leads to formation of a compound of Formula II. Further, the excessive use of alcohol may also cause dryness to the psoriatic skin and irritation to the patients. Thus, the amount of solvent i.e., alcohol used in the present invention remains very critical. The topical composition of the present invention uses alcohol to solubilize halobetasol propionate but does not encompass the amount of alcohol that may cause irritation to the patients or that may lead to formation of the compound with Formula II to an extent that is not acceptable from product quality perspective.

The topical pharmaceutical composition of halobetasol propionate can be any topical dosage form. It can be a solution, spray, gel, lotion, cream, ointment, foam, emulsions (e.g., water-in-oil, water-in-oil-in-water, oil-in-water, silicone-in-water, water-in-silicone, oil-in-water-in-oil, oil-in-water-in-silicone emulsion), microemulsion, nanoemulsion, emulgel, paste, or any other topical composition known in the art. The topical pharmaceutical composition of halobetasol propionate can be presented in any convenient consistency for ease of application. The topical pharmaceutical composition of halobetasol propionate can be prepared in any convenient manner by any method known in the art. Particularly, the topical pharmaceutical composition is a solution. More particularly, the topical pharmaceutical composition is a solution delivered in the form of a spray. Sprays are easy to apply, non-irritant, quick drying and provide enhanced patient compliance. The topical spray composition of halobetasol propionate can be pressurized system i.e., with a propellant (aerosol based) or non-pressurized system i.e., without a propellant. Preferably, the topical spray composition is a pressurized system.

The pressurized topical spray composition of halobetasol comprises halobetasol propionate, a non-aqueous solvent, and a propellant, and one or more pharmaceutically acceptable excipients. Particularly, the pressurized topical spray composition of halobetasol consists essentially of halobetasol propionate, a non-aqueous solvent, and a propellant, and an emollient.

The non-pressurized topical spray composition of halobetasol comprises halobetasol propionate, a non-aqueous solvent, and one or more pharmaceutically acceptable excipients. Particularly, the non-pressurized topical spray composition of halobetasol consists essentially of halobetasol propionate, a non-aqueous solvent, and an emollient.

The pharmaceutically acceptable excipients are added to facilitate the compounding and application of the composition and these are selected from the group consisting of emollients, solubilizers, thickening agents, chelating agents, antioxidants, pH-adjusting agents, permeation enhancers, film-formers, and mixtures thereof.

The term “propellant,” as used herein refers to the substance that helps in propelling the composition out of the container. Suitable examples of propellants are selected from the group consisting of conventional, non-ozone depleting hydrocarbon propellants. These include propane n-butane, isobutane, cyclopropane, liquefied petroleum gas, 1,1,1,2-tetrafluorethane, 1,1,1,2,3,3,3-heptafluoropropane, 1,1-difluoroethane, 1,1,1,3,3,3-hexafluoropropane, or mixtures thereof. Fluorocarbon gas may also be used as a propellant. The propellant is used in the present invention ranges from about 5% w/w to about 95% w/w based on the total weight of the composition. Particularly, the propellant is used in the present invention ranges from about 20% w/w to about 80% w/w based on the total weight of the composition. More particularly, the propellant is used in the present invention ranges from about 30% w/w to about 70% w/w based on the total weight of the composition.

The term “emollient,” as used herein refers to a substance that helps to retain the skin moisture and also helps to control the rate of evaporation and the tackiness of the composition. It also helps to soften the thickened epidermis of the psoriatic plaques. Additionally, emollients provide a softening or soothing effect on the skin surface. Suitable examples of emollients are selected from the group consisting of fatty acid triglycerides such as mixture of caprylic and capric triglycerides (Crodamol™ GTCC-LQ, Miglyol™, Captex™, Labrafac™, Lipophile™ WL), palmitic triglyceride, oleic triglyceride, caprylic triglyceride, capric triglyceride, and linoleic triglyceride; fatty acid esters such as isopropyl myristate, isopropyl palmitate, dibutyl adipate, and dibutyl phthalate; polyhydric alcohols such as propylene glycol, butylene glycol, polyethylene glycol, glycerol, and sorbitol; fatty acids such as oleic acid and stearic acid; oils such as mineral oil, lanolin oil, coconut oil, cocoa butter, olive oil, jojoba oil, and castor oil; cyclomethicone; hydrogenated lanolin; waxes; lecithin; or mixtures thereof. Preferably, the emollient of the present invention is selected from the group consisting of fatty acid triglycerides, fatty acid esters, and polyhydric alcohols. More preferably, the emollient of the present invention is isopropyl palmitate. The emollient used in the present invention ranges from about 1% w/w to about 45% w/w based on the total weight of the composition. Particularly, the emollient used in the present invention ranges from about 1% w/w to about 30% w/w based on the total weight of the composition. More particularly, the emollient used in the present invention ranges from about 1% w/w to about 20% w/w based on the total weight of the composition. Even more particularly, the emollient used in the present invention ranges from about 1% w/w to about 10% w/w based on the total weight of the composition. Even more particularly, the emollient used in the present invention ranges from about 6% w/w to about 8% w/w based on the total weight of the composition.

Suitable solubilizers are selected from the group consisting of polyhydric alcohols such as propylene glycol and polyethylene glycol; fatty acids such as oleic acid and stearic acid; non-ionic and ionic surfactants such as cationic, anionic, and zwitterionic emulsifiers such as polyoxyethyl-sorbitan-fatty acid esters such as polysorbates, ethers of sugars, ethoxylated fatty alcohols, sodium lauryl sulfate, taurocholic acid, lecithin and Labrasol™; vitamin E; vitamin E TPGS (tocopheryl polyethylene glycol 1000 succinate); caprylic triglyceride; capric triglyceride; and mixtures thereof.

Suitable thickening/gelling agents are selected from e.g., polymers, for example, selected from the group consisting of carboxyvinyl polymers such as carbomers (for example, carbomer 940, 971, 973, 974, 980, 981, 941, 974, 934 and 910), celluloses such as hydroxypropyl cellulose, hydroxyethyl cellulose, hydroxypropyl methylcellulose, hydroxypropyl methylcellulose phthalate, methyl cellulose; gums such as xanthan gum, acacia, tragacanth, sodium alginate; gelatin; modified starches; glycols such as propylene glycol, polyethylene glycols; co-polymers formed between maleic anhydride and methyl vinyl ether; methacrylate derivatives; polyethylene oxides; polyoxyethylene-polyoxypropylene copolymers; polyvinyl alcohol; soft paraffin; aluminum stearate; cetostearyl alcohol; hydrogenated lanolin; beeswax; and mixtures thereof.

Suitable chelating agents are selected from the group consisting of dimercaprol, ethylene diamine tetra acetic acid (EDTA), disodium edetate, ethylene glycol tetraacetic acid, alfa lipoic acid; and mixtures thereof.

Suitable antioxidants are selected from the group comprising butylated hydroxyanisol (BHA), butylated hydroxytoluene (BHT), cetyl cysteine, ascorbic acid, esters and derivatives of ascorbic acid, tocopherol and its derivatives, hydroquinone, t-butyl hydroquinone, cysteine HCl, diamylhydroquinone, edetate disodium, edetate trisodium, propyl gallate, octyl gallate, gallic acid esters, isooctyl thioglycolate, kojic acid, sodium bisulfite, sodium metabisulfite, sodium sulfite, superoxide dismutase, sodium thioglycolate, and mixtures thereof.

Suitable pH adjusting agents are selected from the group comprising citric acid, sodium citrate, acetic acid, sodium acetate, phosphoric acid, sodium phosphate, borax, sodium hydroxide; and mixtures thereof.

Suitable permeation enhancers are selected from the group comprising polyols and esters such as polyethylene glycol, polyethylene glycol monolaurate, and butanediol; sulfoxides, such as dimethylsulfoxide and decylmethylsulfoxide; ethers such as diethylene glycol monoethyl ether and diethylene glycol monomethyl ether; fatty acids such as lauric acid, oleic acid, and valeric acid; fatty acid esters such as isopropyl myristate, isopropyl palmitate, methyl propionate, and ethyl oleate; nitrogenous compounds such as urea, dimethyl acetamide, dimethylformamide 2-pyrrolidone, ethanolamine, methyl-2-pyrrolidone, diethanolamine, and triethanolamine; terpenes; alkanones; organic acids such as salicylic acid, citric acid, and succinic acid; surfactants; and mixtures thereof.

The dispensing system for delivering the pressurized system of the present invention comprises a container and a valve-actuator assembly. The valve-actuator assembly may comprise of a valve, a spring, a gasket, a dip tube, an actuator, and a dust cap. Various types of valves such as continuous spray valves and metering valves can be used. The metered valve dispenses a metered quantity of formulation with each actuation of the actuator. The metered quantity avoids under-dosing or overdosing that may lead to undesirable side effects. A dust cap is fitted onto the container to shield the contents of the container from the outside environment.

The dispensing system for delivering the non-pressurized system of the present invention comprises of a container and a pump-actuator assembly. The pump-actuator assembly may comprise of a spring, a gasket, a dip tube, a pump dispenser, a chamber, a dust cap, and an actuator. The pump dispenser dispenses the composition through a dip tube into a chamber. The composition is then dispensed through the actuator with an orifice in the form of a substantially uniform spray or fine droplets. The pump-actuator assembly comprise of a metered pump. The metered pump dispenses a metered quantity with each actuation of the actuator. The metered quantity can avoid under-dosing or overdosing that may lead to undesirable side effects. A dust cap is fitted onto the container to shield the contents of the container from the outside environment.

Containers can be made up from metal such as stainless steel or aluminum, plastic, or glass. The plastic container can be made up of high density polyethylene (HDPE), medium density polyethylene (MDPE), or low density polyethylene (LDPE). The containers made up from metals can be further coated with an inert inner lining of epoxy-phenolic resins, epoxy-urea-formaldehyde resins, polytetrafluroethylene (PTFE), perfluoroethylenepropylene (PFEP), perfluoroalkoxyalkane (PFA), ethylene tetrafluoroethylene (ETFE), polyvinylidene fluoride (PVDF), chlorinated ethylene tetrafluoroethylene, or other coating treatment that creates a barrier to chemical interaction between the composition and the container.

The amount of halobetasol propionate may depend upon the purpose for which the composition is to be applied. For example, the dosage and frequency of application can vary depending upon the type and severity of the topical condition.

The following examples represent various embodiments according to the present invention. The examples are given solely for the purpose of illustration and are not to be construed as limitations of the present invention, as many variations thereof are possible without departing from the spirit and scope of the invention.

### Example

### Example 1: Preparation of ethyl (6α,11β,16β,17Z)-6,9-difluoro-11-hydroxy-16-methyl-3-oxopregna-1,4,17-trien-21-oate (Compound of Formula II wherein R is ethyl)

Halobetasol propionate (5.0 g) was dissolved in ethanol (150 mL) to obtain a solution. The solution was reflux by heating at 80° C. to 120° C. under pressure for one week to obtain a title compound.

Yield: 0.7 g

Mass: 421.4 [M+H]⁺

¹H NMR (CDCl₃, 400 MHz): δ: 1.19 (s, 3H), 1.28 (m, 4H), 1.34 (d, 3H), 1.53-1.62, (m, 4H), 1.67-1.90 (m, 4H), 2.03-2.10 (m, 1H), 2.37-2.53 (m, 3H), 3.30 (m, 1H), 4.16 (q, 2H), 4.36 (m, 1H), 5.33-5.50 (m, 2H), 6.38 (dd, 1H), 6.44 (s, 1H), 7.17 (dd, 1H)

IR_{λmax} (KBr): 3418, 3245, 1710, 1670, 1228, 898, 821 cm⁻¹

The NMR spectrum was recorded using a Bruker® Avance III (400 MHz) NMR spectrometer.

The IR spectrum was recorded using a Perkin Elmer® instrument.

The Mass spectrum was recorded using a MASS (API2000) LC/MS-MS system, Q Trap® LC/MS-MS system (Applied Biosystems).

### Example 2: Preparation of Halobetasol Propionate Spray

| Ingredient | Quantity (% w/w) |
|---|---|
| Halobetasol propionate | 0.05 |
| Ethyl alcohol | 42.65 |
| Isopropyl palmitate | 7.30 |
| Liquefied petroleum gas | 50.00 |

### Procedure:

1. Halobetasol propionate was dissolved into a portion of ethyl alcohol with stirring.
2. Isopropyl palmitate was added while stirring into the solution of step 1.
3. The remaining quantity of ethyl alcohol was added into the solution of step 2 and mixed.
4. The solution of step 3 was filled into an aluminum container with an inert liner, and the container was fitted with a valve assembly.
5. Liquefied petroleum gas was then charged into the container of step 4.

### Stability Data

The halobetasol propionate spray of Example 2 was stored at a temperature of 25° C. and a relative humidity (R.H.) of 60% and at a temperature of 40° C. and a relative humidity (R.H.) of 75% for a period of three months. The spray is analyzed for halobetasol propionate content, a compound of Formula II with R as ethyl, by an ultra-performance liquid chromatography (UPLC) method by using column Acquity UPLC BEH shield RP 18, (100 mm×2.1 mm), 1.7 μm. The results of the analysis are represented in below Tables 1 and 2.

TABLE 1
Stability Data for 3 Months (25° C./60% R.H.)

| | | 3 months | 6 months |
|---|---|---|---|
| Data | Initial | (25° C./60% R.H.) | (25° C./60% R.H.) |
| Assay (%) | 99.7 | 97.8 | 99.5 |
| Compound of | Not | 0.02 | 0.03 |
| Formula II | detected | | |
| with R as ethyl | | | |

TABLE 2
Stability Data for 3 Months (40° C./75% R.H.)

| | | 3 months | 6 months |
|---|---|---|---|
| Data | Initial | (40° C./75% R.H.) | (40° C./75% R.H.) |
| Assay (%) | 99.7 | 97.8 | 99.0 |
| Compound of | Not | 0.15 | 0.35 |
| Formula II | detected | | |
| with R as ethyl | | | |

1. A compound of Formula II 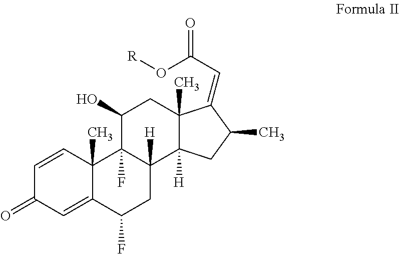
wherein R is C₁-C₄ alkyl.

2. The compound of Formula II of claim 1, wherein R is ethyl.

3. A process for the preparation of a compound of Formula II, comprising reacting halobetasol propionate with a non-aqueous solvent, wherein the Formula II is represented as 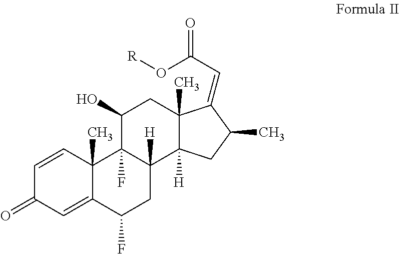
wherein R is C₁-C₄ alkyl.

4. The process for the preparation of claim 3, wherein the compound of Formula II, wherein R is ethyl.

5. The process for the preparation of claim 3, wherein the non-aqueous solvent is selected from the group consisting of methyl alcohol, ethyl alcohol, isopropyl alcohol, n-propyl alcohol, isobutyl alcohol, n-butyl alcohol, or mixtures thereof.

6. The process for the preparation of claim 5, wherein the non-aqueous solvent is ethyl alcohol.

7. A stable topical pharmaceutical composition of halobetasol comprising halobetasol propionate and a compound of Formula II, wherein the content of a compound of Formula II is not more than 2.0% w/w based on the total weight of the composition, and wherein the Formula II is represented as 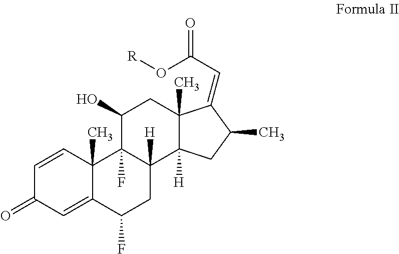
wherein R is C₁-C₄ alkyl.

8. The stable topical pharmaceutical composition of claim 7, wherein the compound of Formula II, wherein R is ethyl.

9. A stable topical pharmaceutical composition of halobetasol comprising halobetasol propionate, a non-aqueous solvent, and a compound of Formula II, wherein the content of a compound of Formula II is not more than 2.0% w/w based on the total weight of the composition, and wherein the Formula II is represented as 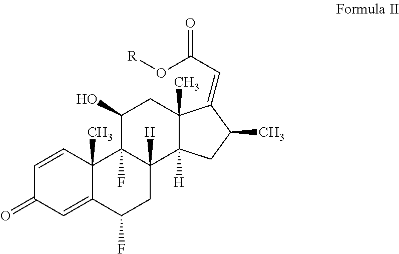
wherein R is C₁-C₄ alkyl.

10. The stable topical pharmaceutical composition of claim 9, wherein the compound of Formula II, wherein R is ethyl.

11. The stable topical pharmaceutical composition of claim 9, wherein the non-aqueous solvent is selected from the group consisting of methyl alcohol, ethyl alcohol, isopropyl alcohol, n-propyl alcohol, isobutyl alcohol, n-butyl alcohol, or mixtures thereof.

12. The stable topical pharmaceutical composition of claim 11, wherein the non-aqueous solvent is ethyl alcohol.

13. The stable topical pharmaceutical composition of claim 9, wherein the non-aqueous solvent is present in an amount of 10% w/w to about 60% w/w based on the total weight of the composition.

14. The stable topical pharmaceutical composition of claim 9, wherein the halobetasol propionate is present from about 0.01% w/w to about 0.5% w/w based on the total weight of the composition.

15. The stable topical pharmaceutical composition of claim 9, wherein the composition is stored at 25° C. and 60% Relative humidity for at least 3 months.

16. The stable topical pharmaceutical composition of claim 9, wherein the composition is stored at 45° C. and 75% Relative humidity for at least 3 months.
